(19) United States
(12) Patent Application Publication
(10) Pub. No.: US 2016/0367440 A1
(43) Pub. Date: Dec. 22, 2016

(54) METHOD FOR ON-SITE COMPOUNDING

(71) Applicant: Prescriber's Choice, LLC, Pompano Beach, FL (US)

(72) Inventor: Alex Chervinsky, Boca Raton, FL (US)

(21) Appl. No.: 15/185,084

(22) Filed: Jun. 17, 2016

Related U.S. Application Data
(60) Provisional application No. 62/181,326, filed on Jun. 18, 2015.

(51) Int. Cl. A61J 3/00; G06F 19/00; A61K 9/00; A61K 31/56; A61K 45/06

(52) U.S. Cl. CPC A61J 3/00; A61K 31/56; A61K 45/06; A61K 9/0014; G06F 19/3456

(57) ABSTRACT

Described is a method for mixing, reconstituting, or compounding a prescription medication “on-site” at a physician's office for on-site or remote administration to the patient.

### BACKGROUND OF THE INVENTION

In the field of prescription medicine, patient compliance—timely filling the prescription, and taking the prescribed dose at the scheduled administration time—has been a continual and prevalent problem. Patient compliance can be especially problematic for compounded medicines, which typically require a compounding pharmacist to prepare an individualized or special-order prescription pursuant to the direction of the doctor. Generally this requires the patient or provider to submit the prescription to a specialty pharmacy that specializes in compounding medications. Most patients are unfamiliar with such specialty pharmacies resulting in further decreases in patient compliance.

“Compounding” has been defined as:

the professional act by a pharmacist or other practitioner authorized by law, employing the science or art of any branch of the profession of pharmacy, incorporating ingredients to create a finished [medicinal] product for dispensing to a patient or for administration by a practitioner or the practitioner's agent; and shall specifically include the professional act of preparing a unique finished product containing any ingredient or device [ . . . ].

Interestingly, the preparation of drugs or devices for sale or transfer to pharmacies, practitioners, or entities for purposes of dispensing or distribution does not always fall within the definition of “compounding” or within the practice of the profession of pharmacy. The pharmacy regulations of many States in the United States prohibit the provision of compounded medications prepared in advance to physicians, which limits the capability of the physician to provide and administer a compounded drug to a patient in the practitioner's office or health care facility or treatment setting, including a hospital, ambulatory surgical center, or pharmacy.

Therefore, a compounding pharmacy or pharmacist cannot supply prepared medicines to a physician so that the physician or authorized prescriber can write, fill, and/or administer the compounded prescription at the point of care, e.g., “on site” at the physician's clinic or may provide the compounded medication to the patient for administration by the patient at home or a remote location at the direction of the physician. For purposes of the subject invention, a compounded medication which is compounded at the physician's office/clinic is referred to as “on-site” compounding.

Thus, while office use compounding could alleviate certain of the issues of patient compliance by having ready-made compounded medications available for prescription sale, filling, and administration in the presence of the physician or health-care worker “on-site,” such practice is at risk of running afoul of State pharmacy regulations.

Thus there is a need for a method for carrying out “on-site” preparation of a compounded medication which can provide the advantages of increased patient compliance while remaining within promulgated pharmacy regulations.

### BRIEF SUMMARY OF THE INVENTION

The subject invention pertains to a method for mixing, reconstituting, or compounding a prescription medication “on-site,” by a physician or authorized compounder, at a physician's clinic comprising the steps of:
a. providing a first prescription medication composition contained within in a first discrete sealed container or package;
b. providing at least a second composition contained within in a second discrete sealed container or package;
   whereby the first and second compositions are activated and compounded for administration by breaching each of the sealed containers followed by mixing, reconstituting, or compounding by the physician or at the direction of the physician at the location where the patient examination is conducted for administration on-site, or for administration by the patient at home or other remote location by the patient.

The term “on-site” means at the clinic or doctor's office or any location where the physician or authorized compounder, and patient are present.

### DETAILED DESCRIPTION OF THE INVENTION

The subject invention relates to a method for mixing, reconstituting, or compounding a prescription medication “on-site” by a physician or by an authorized compounder at the physician's direction, at a physician's clinic, wherein certain components of the compounded medication are provided separately in a plurality of sealed containers and activated “on-site” by the physician or authorized compounder, by breaching each of the respective sealed containers, wherein the compositions mix together to form the compounded medication. The compounded medication can be dispensed as prescribed.

More particularly, a preferred embodiment of a method according to the subject invention comprises the steps of:
a) providing a first prescription medication composition contained within in a first discrete sealed container or package; and
b) providing at least a second composition contained within in a second discrete sealed container or package;
   whereby the first and second compositions are activated and compounded for administration by breaching each of the sealed containers followed by mixing, reconstituting, or compounding by the physician at the location where the patient examination is conducted. The compounded medication can then be dispensed and sold to the patient for administration “on-site” at the clinic or at home or other remote location by the patient.

The first and second compositions can each be selected from a single-component composition, e.g., a substantially pure pharmaceutically acceptable diluent, solvent or carrier, or can comprise a plurality of components, e.g., a single active pharmaceutical ingredient (API) in a substantially pure pharmaceutically acceptable carrier, diluent, or solvent, a plurality of API's in a substantially pure pharmaceutically acceptable carrier, diluent, or solvent, or one or more API in a pharmaceutically acceptable carrier, diluent, or solvent wherein the carrier, diluent, or solvent comprises a plurality of ingredients or components, each of which can be substantially pure.

In addition, the number of separate containers can be two or more, e.g., two, three, four, or more, each containing a composition for mixing together and forming a single compounded medication when dispensed.

The physician or other authorized health care worker can activate each of the containers by breaching the respective seals, thereby allowing the separate compositions to mix together and become “compounded,” and thereafter administer the compounded medication to the patient.

The method of the invention can further include one or more of the steps of:
writing a prescription for the compounded medication,
selling the compounded medication, and
delivering the compounded medication to the patient for immediate administration or for later use by the patient.

One preferred method of the invention is to provide the first and second fluid compositions in separately sealed containers wherein the seal of both containers is breached to allow each of the compositions to be in communication with a single dispenser. The composition from the first container and the second container can thereby be administered together as a compounded mixture from the single dispenser. A fluid composition useful for purposes of the subject invention can be a liquid, such as a single-ingredient in liquid form, a solution or suspension, a gel, a semi-solid, a solid suspension, or other composition having fluid properties and capable of being dispensed from its container.

It should be understood that the separate containers for the respective first and second compositions can be provided separately as individual containers, can be provided as separate containers that are capable of being adjoined to form a single unitary container, or can be provided as a single container having two or more discrete chambers, so long as the first and second compositions are discretely packaged and sealed, and are not mixed, reconstituted, or otherwise compounded prior to an activation step.

In one preferred embodiment, activation can be effected by breaching the seal of both first and second containers using a single dispenser which has an access port for communicating simultaneously, but separately, with each of the compositions. In the case of fluid compositions, the dispenser can have a nozzle which regulates the flow of the mixed and compounded compositions prior to or when administered.

The dispenser can further comprise a mixing chamber which allows the first and second compositions to come together at a particular desired ratio prior to dispensing from the nozzle and can further be adjustable so that the ratio of compositions from the first and second (or third, or fourth as the case may be) containers or chambers can be selectively modified as desired.

One preferred embodiment of the invention comprises a device which precisely compounds medications for individual patient needs by selectively mixing the first and second compositions in a predetermined ratio by use of a dual dispensing device. In one embodiment of the invention, the dual dispensing device can be capable of selectively adjusting and customizing the ratio of the first and second compositions to be mixed or compounded prior to administration from the dispenser. Such devices, are commercially available, for example, from VariBlend (Charleston, S.C., USA) as described and disclosed at www.variblend.com, the contents and descriptions of which are hereby incorporated by reference in their entirety.

Automated mixing and dispensing devices can also be used in accordance with the subject invention. One example of an automated mixing and dispensing device useful for mixing and dispensing the components to be compounded is the Sympress Dispenser which is commercially available from Kettenbach GmbH & Co. KG (Eschenburg, Germany). Such automated dispensers are primarily sold for mixing and dispensing dental impression material contained within foil bag cartridges, but may be adapted by a person of ordinary skill in the art for mixing and dispensing materials for compounding a compounded medication.

The use of an automated dispenser can advantageously provide accurate mixing, dispensing and application with repeatable material properties, including: homogenous, bubble free mixing; selectable extrusion speed; and automatic propulsion and reverse drive. These features can provide benefits to the user such as ease of mixing, hygienic mixing, consistent results, and economy of materials required.

It would be understood that the dispensing device can be non-adjustable, to provide be a predetermined, fixed ratio of the compositions, and alternatively can have the capability of being adjustable for providing different ratios as desired by the physician or compounder. The dispenser may also be adapted to mix three or more compositions from respectively three or more containers or chambers.

In carrying out the method of the invention, the compositions can comprise any pharmaceutically or cosmetically acceptable ingredient.

### EXAMPLE

One embodiment of a method according to the subject invention comprises on-site compounding of a topical dermal composition by a physician. A patient presents at a dermatologist's office with atopic dermatitis, which the physician seeks to treat using a compounded mixture of a 5% corticosteroid preparation and a 10% antibacterial preparation, mixed at a 10:1 ratio.
Step 1: The physician writes a prescription for compounding a medicated preparation comprising 5% corticosteroid and 10% antibacterial preparation.
Step 2: The corticosteroid preparation and antibacterial preparation are provided in separate, prepared, sealed containers, each container capable of being activated contemporaneously using a single dispenser device.
Step 3: The physician selects a single dispenser device that provides the capability for each of the compositions from each of the separate containers to be activated (mixed or compounded) and dispensed, or activated and mixed together within the dispenser, at a predetermined 10:1 ratio. Alternatively, a dispenser device that can selectively be adjusted to dispense, or allow the mixture and compounding of, the compositions from each of the separate containers at a 10:1 ratio.
Step 4: The user of the mixing and dispensing device, such as the physician or an authorized compounder, activates each of the discrete containers by affixing a single dispenser to each of the separate and sealed containers, breaching the seal of each container ((i.e., “compounding” the prescribed compounded medication) whereby the compositions in each of the containers can be dispensed, or allowed to mix together and then dispensed, at the predetermined ratio.
Step 5: The physician who prepares the compounded medication can then optionally administer the composition, or can deliver the compounded medication to the patient for later use, or both.

Each of the above steps can be carried out on-site in the presence of the patient.

The advantages of the method according to the subject invention are manifold. For example, the physician can compound prescription medications on-site and treat the patient in need immediately or can provide the compounded medication to the patient for later administration at home or other remote location at the direction of the physician and in accordance with the dosing schedule prescribed.

In addition, the patient compliance can be increased because the risk of not having the prescription filled by a compounding pharmacist, the risk of incorrect usage, application, or administration of the medication, the risk of delayed treatment by the patient forgetting administration of the medication, and the like, are all minimized by the described on-site compounding method.

Another advantage of the subject method of on-site compounding is that the physician can demonstrate or provide full explanation to the patient regarding the optimal administration or application process for the compounded medication.

A further advantage of the described method is that the physician and compounding pharmacist can effectively and timely provide treatment to patients without violating compounding pharmacy regulations.

Other and additional advantages would be readily recognized by persons of ordinary skill in the compounded medication art.

While the foregoing has been set forth in detail, it is to be understood that the embodiments are presented for elucidation and not limitation. Design variations, especially in matters of shape, size and arrangements of parts of the device or devices employed in the method may be made but are within the scope and spirit of the principles described herein. Those skilled in the art will realize that such changes or modifications of the invention or combinations of elements, variations, equivalents or improvements therein are still within the scope of the mixing and dispensing device as described in the specification and defined in the appended claims.

1. A method for on-site mixing, reconstituting, or compounding a prescription medication by a physician, authorized compounder wherein the method comprises the steps of:
a. providing a plurality of components for forming a compounded medication, each component in a separate, sealed container;
b. activating each component in each container by breaching each of the sealed containers using a single dispenser, whereby the compositions mix together to form the compounded medication.

2. The method of claim 1 further comprising the step of:
writing a prescription for the compounded medication or the components thereof.

3. The method of claim 1 further comprising the step of:
selling the compounded medication or the components thereof.

4. The method of claim 1 further comprising the step of:
delivering the compounded medication to the patient for immediate or later use by the patient.

5. The method of claim 1 wherein the prescribed compounded medication is dispensed.

6. The method of claim 2 wherein the prescribed compounded medication is administered to the patient in need of the compounded medication.

7. A method for on-site mixing, reconstituting, or compounding a prescription medication by a physician or authorized compounder wherein the method comprises the steps of:
a. providing a first prescription medication composition contained within in a first discrete sealed container or package; and
b. providing at least a second composition contained within in a second discrete sealed container or package;
whereby the first and second compositions are activated and compounded for administration by breaching each of the sealed containers followed by mixing, reconstituting, or compounding by the physician at the location where the patient examination is conducted.

8. The method of claim 1 wherein the mixing of the compounding materials is carried out using an automated mixing and dispensing device.
